(19) United States
(12) Patent Application Publication
(10) Pub. No.: US 2019/0082935 A1
(43) Pub. Date: Mar. 21, 2019

(54) VARIABLE RIGIDITY APPARATUS

(71) Applicant: OLYMPUS CORPORATION, Tokyo (JP)

(72) Inventor: Tomohiro KITANAKA, Higashimurayama-shi (JP)

(73) Assignee: OLYMPUS CORPORATION, Tokyo (JP)

(21) Appl. No.: 16/195,913

(22) Filed: Nov. 20, 2018

Related U.S. Application Data
(63) Continuation of application No. PCT/JP2017/000667, filed on Jan. 11, 2017.

(30) Foreign Application Priority Data
May 25, 2016 (JP) PCT/JP2016/065453

(51) Int. Cl. A61B 1/005

(52) U.S. Cl. CPC A61B 1/0055; A61M 25/0138; A61B 1/0057

(57) ABSTRACT

A variable stiffness apparatus is installed inside a flexible member to provide different levels of stiffness to the flexible member. The variable stiffness apparatus includes at least one variable stiffness unit. Each of the at least one variable stiffness unit includes a flexible coil pipe, a core wire extending inside the coil pipe, a pair of fixing members arranged on both sides of the coil pipe and fixed to the core wire, and an adjustment mechanism that adjusts at least one gap between the coil pipe and at least one of the fixing members.

### CROSS-REFERENCE TO RELATED APPLICATIONS

This application is a Continuation Application of PCT Application No. PCT/JP2017/000667, Jan. 11, 2017 and based upon and claiming the benefit of priority from the prior PCT Application No. PCT/JP2016/065453, filed May 25, 2016, the entire contents of which are incorporated herein by reference.

### BACKGROUND OF THE INVENTION

### 1. Field of the Invention

The present invention relates to a variable stiffness apparatus that changes the stiffness of a flexible member in which the variable stiffness apparatus is installed.

### 2. Description of the Related Art

Japanese Patent No. 3122673 discloses an endoscope capable of changing the stiffness of a flexible section of an insertion section. In this endoscope, both ends of a coil pipe are fixed at predetermined positions in the endoscope, and a flexibility adjustment wire inserted through the coil pipe is fixed to the coil pipe through a separator. The coil pipe and the flexibility adjustment wire extend to a control section along the flexible section, and extend through almost the entire flexible section. The coil pipe is compressed and stiffened by pulling the flexibility adjustment wire, thereby changing the stiffness of the flexible section.

### BRIEF SUMMARY OF THE INVENTION

The present invention is directed to a variable stiffness apparatus that is installed inside a flexible member to provide different levels of stiffness to the flexible member. The variable stiffness apparatus includes at least one variable stiffness unit. Each of the at least one variable stiffness unit includes a flexible coil pipe, a core wire extending inside the coil pipe, a pair of fixing members arranged on both sides of the coil pipe and fixed to the core wire, and an adjustment mechanism that adjusts at least one gap between the coil pipe and at least one of the fixing members.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 shows a basic configuration of a variable stiffness apparatus according to a first embodiment.

FIG. 2 shows a configuration example of an adjustment mechanism of the variable stiffness apparatus according to the first embodiment.

FIG. 3 shows a change in state of the variable stiffness unit from a straight state when the bending stiffness is high as shown in the lower part of FIG. 1 to a bent state.

FIG. 4 shows a variable stiffness unit according to a second embodiment along with the variable stiffness unit according to the first embodiment.

FIG. 5 shows a state in which the variable stiffness unit of the second embodiment is bent.

FIG. 6 shows a variable stiffness apparatus according to a third embodiment.

FIG. 7 shows the variable stiffness apparatus according to the third embodiment.

FIG. 8 shows a variable stiffness unit according to a fourth embodiment.

FIG. 9 shows a state in which the variable stiffness unit adjusted to the state shown in the lower part of FIG. 8 is gradually bent to increase the bending.

FIG. 10 shows an endoscope in which the variable stiffness unit of the fourth embodiment is installed.

FIG. 11 shows a state in which the variable stiffness unit adjusted to the state shown in the lower part of FIG. 8 is bent, and a coil pipe abuts fixing members through the washer.

FIG. 12 shows a state in which the variable stiffness unit shown in FIG. 11 is bent with different curvature radii.

FIG. 13 shows a variable stiffness unit according to a fifth embodiment.

FIG. 14 shows an enlarged view of a part of the variable stiffness unit shown in FIG. 13.

FIG. 15 shows an enlarged view of a part of the variable stiffness unit shown in FIG. 13.

FIG. 16 shows a variable stiffness unit according to a fifth embodiment in which a sufficient number of gap members are arranged inside a coil pipe.

FIG. 17 shows a variable stiffness unit according to the fifth embodiment provided with an urging member that urges the gap members.

FIG. 18 shows a variable stiffness unit according to a sixth embodiment.

FIG. 19 shows a state in which the variable stiffness unit shown in FIG. 18 is bent in different bending directions.

FIG. 20 shows a state in which the flexible tube of the endoscope is inserted in the large intestine.

FIG. 21 shows a variable stiffness unit according to a seventh embodiment.

FIG. 22 shows a gap member shown in FIG. 21.

FIG. 23 shows a state in which the variable stiffness unit shown in FIG. 21 is bent in different bending directions.

### DETAILED DESCRIPTION OF THE INVENTION

### First Embodiment

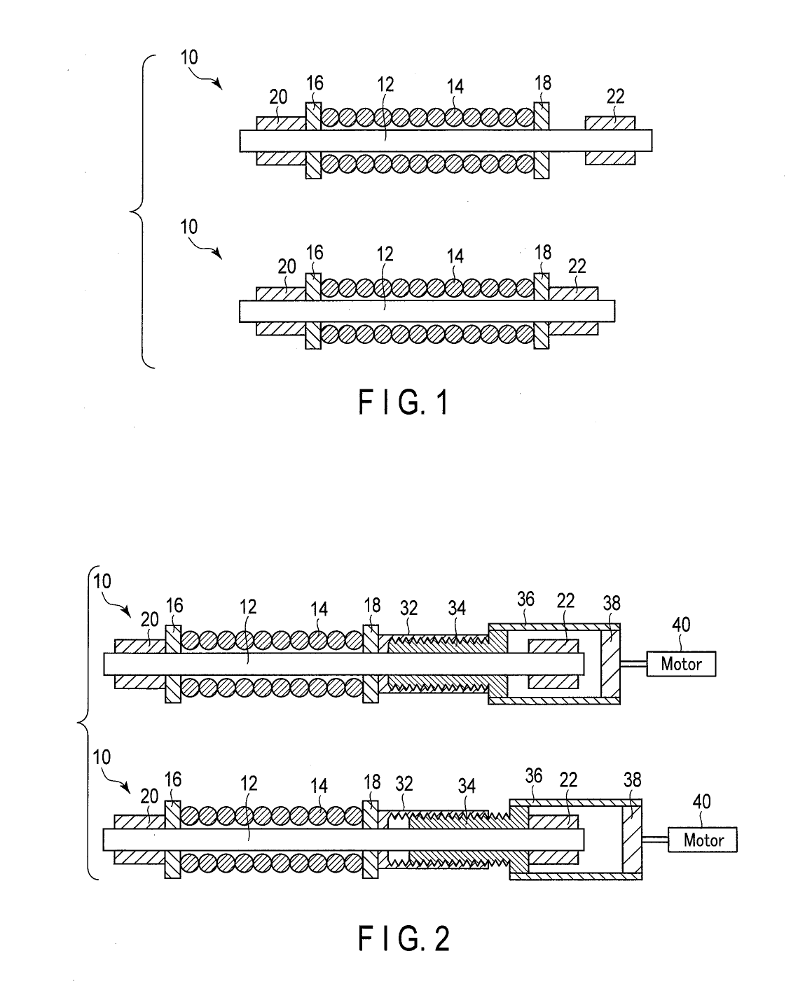

FIG. 1 shows a basic configuration of a variable stiffness apparatus according to a first embodiment. The variable stiffness apparatus is an apparatus that is installed inside a flexible member to provide different levels of stiffness to the flexible member. The variable stiffness apparatus includes at least one variable stiffness unit 10.

The variable stiffness unit 10 includes a flexible coil pipe 14 such as a contact coil, a core wire 12 extending inside the coil pipe 14, and a pair of fixing members 20 and 22 arranged on both sides of the coil pipe 14 and fixed to the core wire 12.

A washer 16 is arranged between the coil pipe 14 and the fixing member 20. A washer 18 is arranged between the coil pipe 14 and the fixing member 22. The washers 16 and 18 serve to restrict the movement of the coil pipe 14 along the core wire 12. The washers 16 and 18 prevent the coil pipe 14 from falling off from the core wire 12, and prevent the fixing members 20 and 22 from digging into the coil pipe 14.

In the variable stiffness unit 10, gaps between the coil pipe 14 and the fixing member 20 or 22 are adjustable. There may be gaps between the coil pipe 14 and both of the fixing members 20 and 22; however, it is assumed that there is only a single gap for convenience hereinafter. To be exact, the gap is a gap between the washer 16 or 18 and the fixing member 20 or 22, but is referred to as a gap between the coil pipe 14 and the fixing member 20 or 22 for convenience hereinafter. The gap between the coil pipe 14 and the fixing member 20 or 22 may also be referred to as play in the axial direction with respect to the core wire 12.

For example, at least one of the fixing members 20 and 22 is releasable from being fixed to the core wire 12, and may be movable along the core wire 12 if released from being fixed. In this case, the at least one of the fixing members 20 and 22 releasable from being fixed constitutes an adjustment mechanism that adjusts at least one gap between the coil pipe 14 and at least one of the fixing members 20 and 22.

In order to obtain the required stiffness, it is preferable that the coil pipe 14 preferably has a length of 20 mm to 500 mm, and the ratio of the outer diameter to the length is 1:2 to 1:50.

In the state shown in the upper part of FIG. 1, there is a gap between the coil pipe 14 and the fixing member 20 or 22, in other words, the core wire 12 has play in the axial direction, and thus the core wire 12 is movable along the coil pipe 14. In this state, a tensile stress is not applied to the core wire 12 when the coil pipe 14 is bent; accordingly, the bending stiffness is low.

In the state shown in the lower part of FIG. 1, there is no gap between the coil pipe 14 and the fixing member 20 or 22, in other words, the core wire 12 has no play in the axial direction, and the core wire 12 is immovable with respect to the coil pipe 14. In this state, a tensile stress is applied to the core wire 12 when the coil pipe 14 is bent; accordingly, the bending stiffness is high. In addition, the fixing members 20 and 22 may be fixed to the core wire 12 with a tensile stress being applied to the core wire 12.

Hereinafter, a state in which the core wire 12 is movable is referred to as a low-stiffness state, and a state in which the core wire 12 is immovable is referred to as a high-stiffness state.

FIG. 2 shows a configuration example of the adjustment mechanism of the variable stiffness apparatus according to the first embodiment. The adjustment mechanism is constituted by a pulling mechanism that pulls at least one of the pair of fixing members 20 and 22 in a direction to move the pair of fixing members 20 and 22 away from each other. This pulling mechanism includes a nut 32, a lead screw 34 screwed to the nut 32, a tube 36 fixed to the lead screw 34, a lid 38 fixed to the tube 36, and a motor 40 that rotates the lead screw 34.

The core wire 12 extends through the nut 32 and the lead screw 34. The fixing member 22 is contained inside the tube 36. The motor 40 is supported so that the motor 40 itself does not rotate and is movable in the axial direction. The lead screw 34 is movable along the axis of the core wire 12 by rotating the lead screw 34 with respect to the nut 32 by the motor 40.

In the state shown in the upper part of FIG. 2, there is a gap between the lead screw 34 and the fixing member 22. In this state, the core wire 12 is movable along the coil pipe 14. In this state, a tensile stress is not applied to the core wire 12 when the coil pipe 14 is bent; accordingly, the bending stiffness is low.

On the other hand, in the state shown in the lower part of FIG. 2, there is no gap between the lead screw 34 and the fixing member 22. In this state, the core wire 12 is immovable with respect to the coil pipe 14. Furthermore, the lead screw 34 presses the fixing member 22, and a tensile stress is applied to the core wire 12. In this state, a tensile stress is further applied to the core wire 12 when the coil pipe 14 is bent; accordingly, the bending stiffness is high.

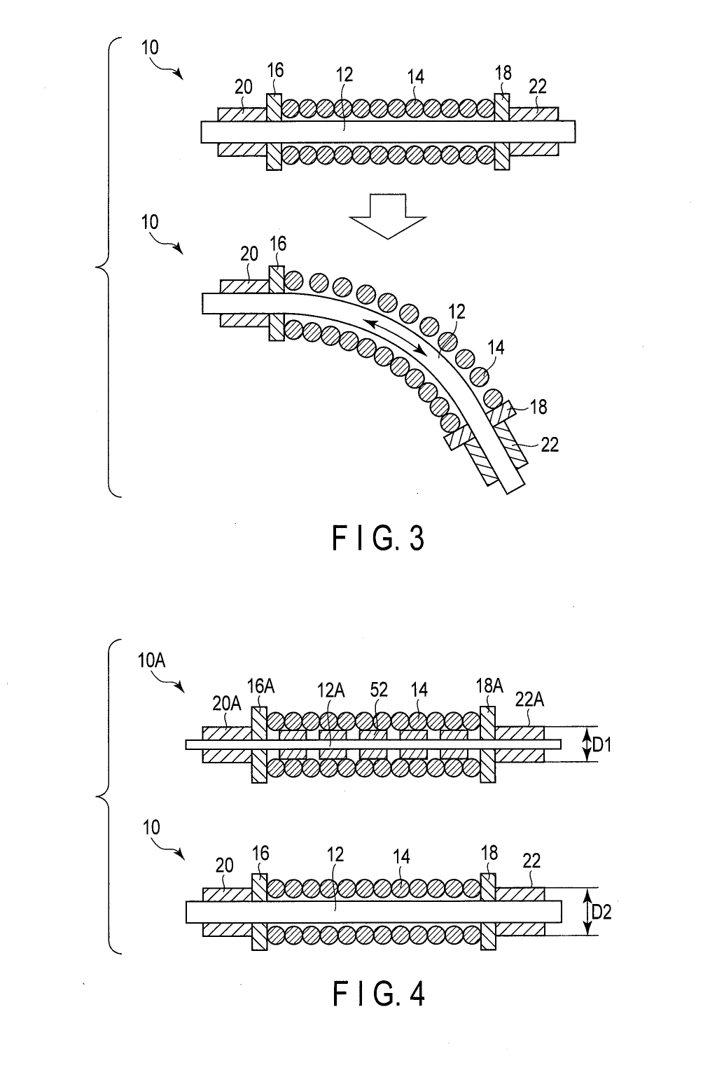

FIG. 3 shows a change in state of the variable stiffness unit 10 from a straight state when the bending stiffness is high as shown in the lower part of FIG. 1 to a bent state. When the coil pipe 14 is bent, the core wire 12 passing through the coil pipe 14 is stretched, which leads to an increase in the bending stiffness of the variable stiffness unit 10. The bending stiffness of the variable stiffness unit 10 increases when the core wire 12 is stretched as the coil pipe 14 is bent and a tensile stress to the core wire 12 increases as the core wire 12 is stretched.

As described above, in the present embodiment, since the high-stiffness state can be obtained only by eliminating the play of the core wire 12 in the axial direction, as described above, it is not necessary to apply a large compression force to the coil pipe 14. The necessity of a compression force decreases as the length of the core wire 12 between the fixing members 20 and 22 decreases.

The present embodiment achieves a simply-configured variable stiffness apparatus that is installed in a flexible member to provide different levels of stiffness to the flexible member.

### Second Embodiment

FIG. 4 shows a variable stiffness unit 10A according to a second embodiment along with the variable stiffness unit 10 according to the first embodiment. Similar to the variable stiffness unit 10 according to the first embodiment, the variable stiffness unit 10A includes a coil pipe 14, a core wire 12A, washers 16A and 18A, and fixing members 20A and 22A.

The core wire 12A of the variable stiffness unit 10A of the present embodiment is made thinner than the core wire 12 of the variable stiffness unit 10 of the first embodiment. Accordingly, the washer 16A or 18A has a through hole with a smaller diameter than the through hole of the washer 16 or 18. The fixing member 20A or 22A has a smaller outer diameter than the fixing member 20 or 22. In other words, the outer diameter D1 of the fixing member 20A or 22A is smaller than the outer diameter D2 of the fixing member 20 or 22. Such fixing members 20A and 22A with a small diameter contribute to miniaturization of an adjustment mechanism that adjusts at least one gap between the coil pipe 14 and at least one of the fixing members 20A and 22A.

The coil pipe 14 of the variable stiffness unit 10A of the present embodiment is the same as the coil pipe 14 of the variable stiffness unit 10 of the first embodiment. This is because the coil pipe 14 needs to have an appropriate thickness in order to obtain the required stiffness.

The variable stiffness unit 10A further includes gap members 52 that maintain a distance between the coil pipe 14 and the core wire 12A when the coil pipe 14 is bent. The gap members 52 each have a pipe shape, and are arranged inside the coil pipe 14 and outside the core wire 12A. The core wire 12A extends through the gap members 52. Each gap member 52 may be made of, for example, a short metal pipe. The length of each gap member 52 is preferably short so as not to affect the stiffness of the entire variable stiffness unit 10A.

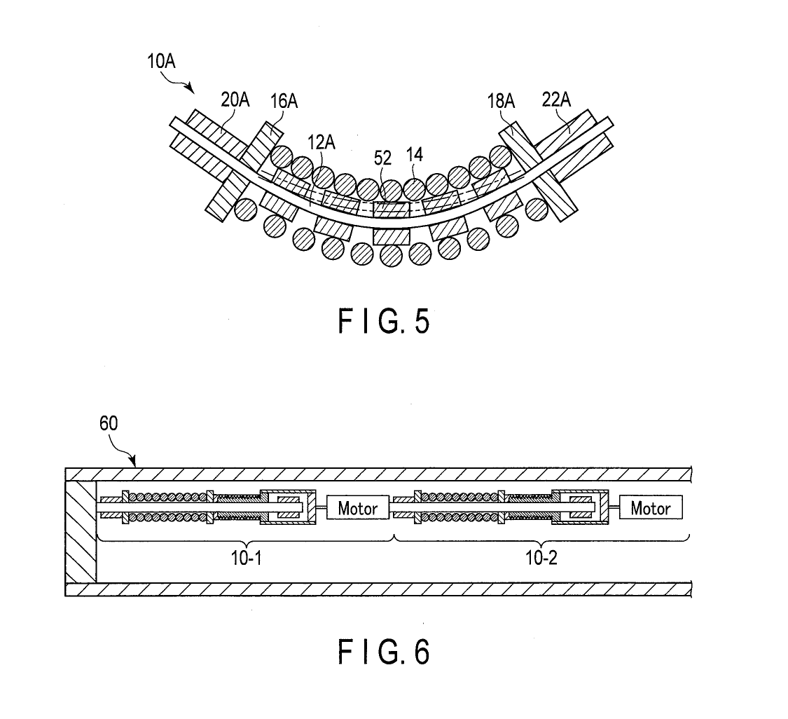

FIG. 5 shows a state in which the variable stiffness unit 10A of the present embodiment is bent. In the absence of the gap members 52, the core wire 12A comes close to the bending center portion, as indicated by the imaginary line. In contrast, if the gap members 52 are provided, the gap members 52 prevent radial movement of the core wire 12A. As a result, the core wire 12A does not come close to the bending center portion, and the distance between the coil pipe 14 and the core wire 12A is thus maintained constant. The curvature of the core wire 12A in the presence of the gap members 52 is larger than the curvature of the core wire 12A in the absence of the gap members 52. Thus, the stretched amount of the core wire 12A in the presence of the gap members 52 is larger than that in the absence of the gap members 52. Therefore, the stiffness of the variable stiffness unit 10A increases.

In the variable stiffness unit 10A of the present embodiment, the core wire 12A is prevented from coming close to the bending center when the coil pipe 14 is bent. As a result, the curvature of the core wire 12A increases, and higher stiffness than the first embodiment is obtained.

Since the stiffness in the state in which the core wire 12A is movable (the low-stiffness state) is the same as the first embodiment, a larger stiffness change quantity than the first embodiment is obtained.

Since the fixing members 20A and 22A have a small diameter, an adjustment mechanism that adjusts at least one gap between the coil pipe 14 and at least one of the fixing members 20A and 22A can be made small.

Also in the present embodiment, it is not necessary to apply a large compression force to the coil pipe 14.

### Third Embodiment

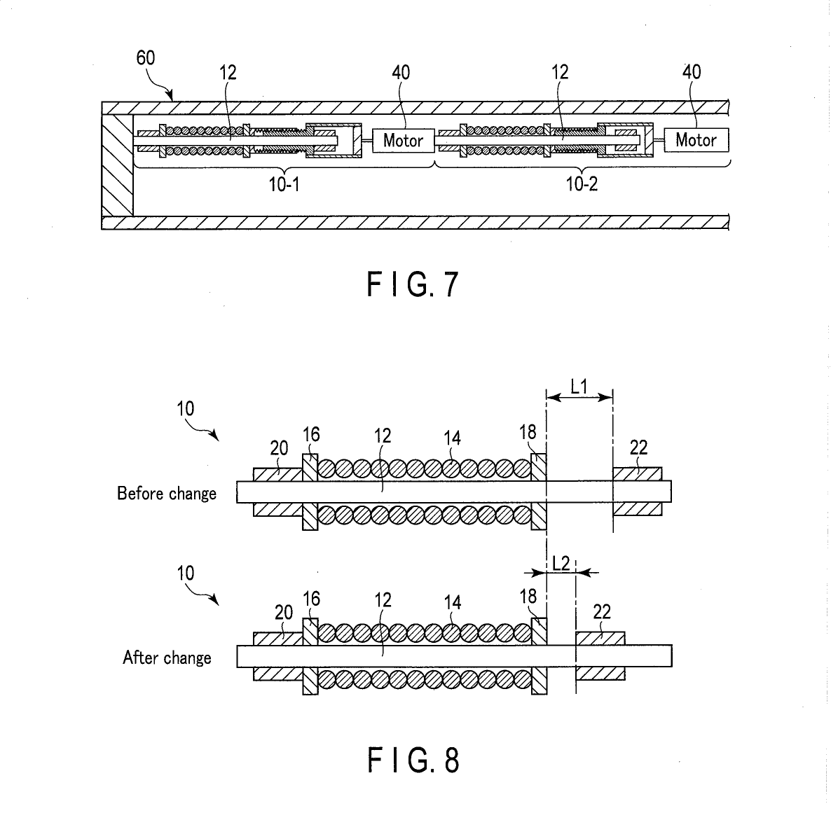

FIGS. 6 and 7 show a variable stiffness apparatus according to a third embodiment. As shown in FIGS. 6 and 7, the variable stiffness apparatus includes variable stiffness units 10-1 and 10-2 arranged inside a flexible member such as a flexible tube 60 along a longitudinal direction. To each of the variable stiffness units 10-1 and 10-2, the variable stiffness unit 10 or 10A of the first embodiment or the second embodiment may be applied. FIG. 6 and FIG. 7 illustrate two variable stiffness units 10-1 and 10-2; however, the number of the variable stiffness units 10-1 and 10-2 is not limited thereto. In other words, the variable stiffness apparatus may include three or more variable stiffness units.

In the state shown in FIG. 6, both of the variable stiffness units 10-1 and 10-2 are in the low-stiffness state. Accordingly, the flexible tube 60 is easily bendable in both of the area where the variable stiffness unit 10-1 is arranged and the area where the variable stiffness unit 10-2 is arranged.

In the state shown in FIG. 7, on the other hand, the variable stiffness unit 10-2 is in the low-stiffness state, but the variable stiffness unit 10-1 is in the high-stiffness state. Accordingly, the flexible tube 60 is easily bendable in the area where the variable stiffness unit 10-2 is arranged, but is difficult to be bent in the area where the variable stiffness unit 10-1 is arranged.

In the present embodiment, as described above, the bending stiffness of the flexible tube 60 can be partially changed.

Also in the present embodiment, it is not necessary to apply a large compression force to the coil pipe 14. A core wire 12 of the variable stiffness unit 10-2 is connected to a motor 40 of the variable stiffness unit 10-1, so that the entire variable stiffness unit 10-2 moves in accordance with the axial movement of the motor 40 of the variable stiffness unit 10-1. However, the variable stiffness unit 10-2 can be made independent by separating the core wire 12 of the variable stiffness unit 10-2 from the motor 40 of the variable stiffness unit 10-1. Accordingly, a part of the flexible tube 60 where the bending stiffness is changed can be fixed.

### Fourth Embodiment

FIG. 8 shows a variable stiffness unit according to a fourth embodiment. In the variable stiffness unit 10 of the present embodiment, a gap between the coil pipe 14 and the fixing member 20 or 22 is continuously adjustable. The variable stiffness unit 10 of the present embodiment may be constituted by the variable stiffness unit 10 or 10A of the first embodiment or the second embodiment.

In the state shown in the upper part of FIG. 8, the gap between the coil pipe 14 and the fixing member 20 or 22 is adjusted to be wide. Here, a length L1 of the gap between the coil pipe 14 and the fixing member 20 or 22 is adjusted so that the coil pipe 14 does not abut the fixing member 20 or 22 even if the variable stiffness unit 10 is bent to the maximum expected bending.

On the other hand, in the state shown in the lower part of FIG. 8, the gap between the coil pipe 14 and the fixing member 20 or 22 is adjusted to be narrow. Herein, a length L2 of the gap between the coil pipe 14 and the fixing member 20 or 22 is adjusted so that the coil pipe 14 abuts the fixing member 20 or 22 in the process of bending the variable stiffness unit 10 to the maximum expected bending.

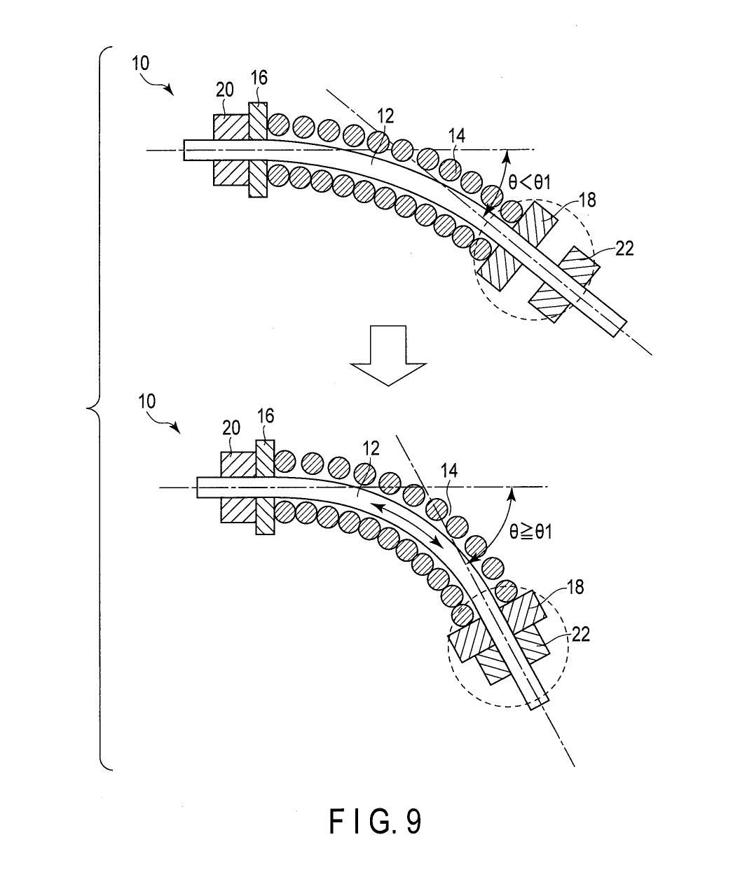

FIG. 9 shows a state in which the variable stiffness unit 10 adjusted to the state shown in the lower part of FIG. 8 is gradually bent to increase the bending. The upper part of FIG. 9 shows a state in which the bending of the variable stiffness unit 10 is relatively small. The lower part of FIG. 9 shows a state in which the bending of the variable stiffness unit 10 is relatively large, and the coil pipe 14 abuts the fixing member 20 or 22 through the washer 18.

In the state shown in the upper part of FIG. 9, the bending angle θ of the core wire 12 is smaller than θ1, and there is a gap between the coil pipe 14 and the fixing member 20 or 22. In other words, the core wire 12 has play in the axial direction. Accordingly, the core wire 12 is movable along the coil pipe 14. In this state, since a tensile stress is not applied to the core wire 12, the bending stiffness is low.

On the other hand, in the state shown in the lower part of FIG. 9, the bending angle θ of the core wire 12 is equal to or greater than θ1, and there is no gap between the coil pipe 14 and the fixing member 20 or 22. In other words, the core wire 12 has no play in the axial direction. Accordingly, the core wire 12 is immovable with respect to the coil pipe 14. In this state, a tensile stress is applied to the core wire 12 when the core wire 12 is further bent, or a tensile stress is already applied to the core wire 12. Accordingly, the bending stiffness is high.

In the present embodiment, as described above, the stiffness of the variable stiffness unit 10 changes with the specific bending angle θ1 as the starting point. Specifically, the variable stiffness unit 10 is in a low-stiffness state if the bending angle θ of the core wire 12 is smaller than θ1, and is in a high-stiffness state if the bending angle θ of the core wire 12 is equal to or greater than θ1. In other words, the stiffness of the variable stiffness unit 10 changes when the core wire 12 is bent at the specific bending angle θ1 or further.

The specific bending angle θ1 at which the stiffness of the variable stiffness unit 10 changes can be changed by changing the length of the gap between the coil pipe 14 and the fixing member 20 or 22. It is thereby possible to limit the bending angle of the flexible member in which the variable stiffness unit 10 is installed.

Also in the present embodiment, it is not necessary to apply a large compression force to the coil pipe 14.

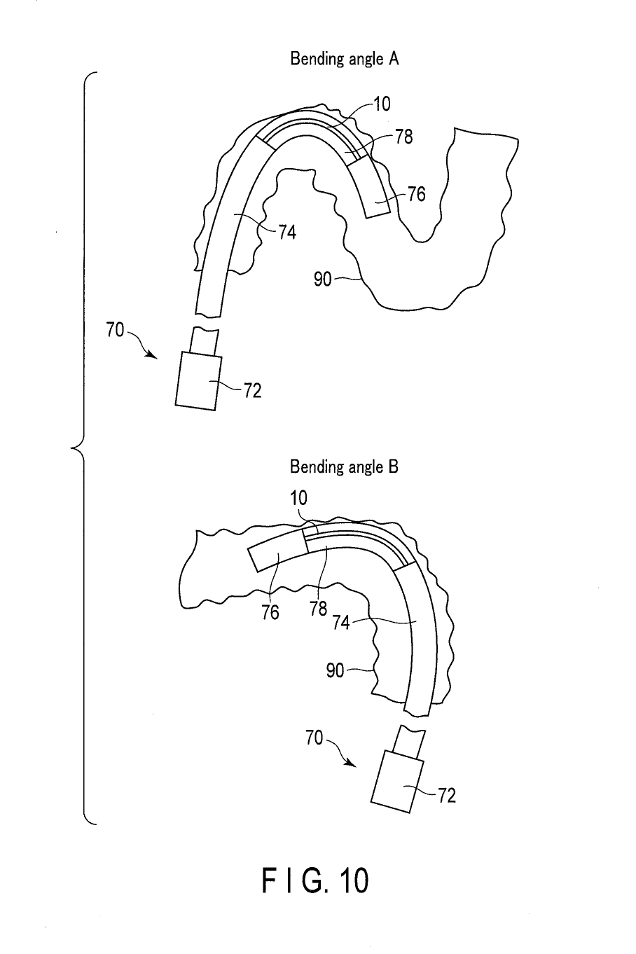

FIG. 10 shows an endoscope 70 in which the variable stiffness unit 10 of the present embodiment is installed. The endoscope 70 includes a grip 72 for the operator to hold the endoscope 70, and a flexible tube 74 extending from the grip 72. The grip 72 is provided with a control section such as a knob, a lever, and a dial. The flexible tube 74 includes an active bendable section 76 that is bendable by operation through the control section of the grip 72, and a passive bendable section 78 that is located closer to the hand side than the active bendable section 76. The variable stiffness unit 10 is provided inside the passive bendable section 78. The variable stiffness unit 10 extends along the passive bendable section 78. The play of the core wire 12 of the variable stiffness unit 10 in the axial direction is changeable by operating the control section of the grip 72.

In the state shown in the upper part of FIG. 10, the play of the core wire 12 of the variable stiffness unit 10 in the axial direction is adjusted so that the passive bendable section 78 is not bent beyond a bending angle A, taking into consideration the shape of a large intestine 90 at the section where the passive bendable section 78 is inserted. In the state shown in the lower part of FIG. 10, the play of the core wire 12 of the variable stiffness unit 10 in the axial direction is adjusted so that the passive bendable section 78 is not bent beyond a bending angle B, taking into consideration the shape of the large intestine 90 at the section where the passive bendable section 78 is inserted.

As described above, it is possible to enhance the insertability of the flexible tube 74 of the endoscope 70 by limiting the bending angle of the passive bendable section 78 in consideration of the shape of the large intestine 90 at the section where the passive bendable section 78 is inserted.

### Fifth Embodiment

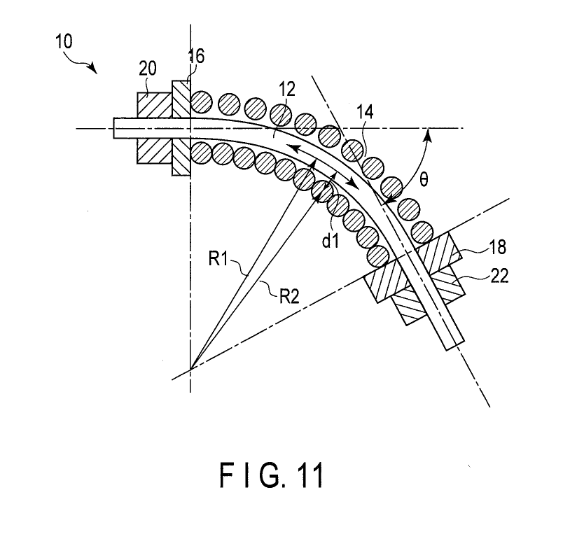

FIG. 11 shows a state in which the variable stiffness unit adjusted to the state shown in the lower part of FIG. 8 is bent, and the coil pipe abuts the fixing member through the washer. In this state, the bending angle θ of the core wire 12 is equal to the specific bending angle θ1 at which the stiffness of the variable stiffness unit 10 changes.

A length L2 of the gap between the coil pipe 14 and the fixing member 20 or 22 when the variable stiffness unit 10 is in a linear state is hereinafter abbreviated as a gap length L2. The difference d1 (=R1−R2) between a curvature radius R1 of the core wire 12 and an inner curvature radius R2 of the coil pipe 14, in other words, a distance d1 from the central axis of the core wire 12 to the center of the cross section perpendicular to the spirally-extending central axis of the wire material of the coil pipe 14 is hereinafter abbreviated as a center-to-center distance d1. Herein, the gap length L2 and the center-to-center distance d1 satisfy the relationship as L2=d1×θ1.

From this relational expression, it is understood that the bending angle θ1 of the core wire 12 when the stiffness of the variable stiffness unit 10 changes, in other words, the bending angle θ1 of the core wire 12 when the variable stiffness unit 10 is stiffened depends on the gap length L2 and the center-to-center distance d1.

The gap length L2 and the center-to-center distance d1 define the bending angle θ1 of the core wire 12 at which the stiffness of the variable stiffness unit 10 changes, but are not related to, for example, the curvature radius R1 of the core wire 12 at all.

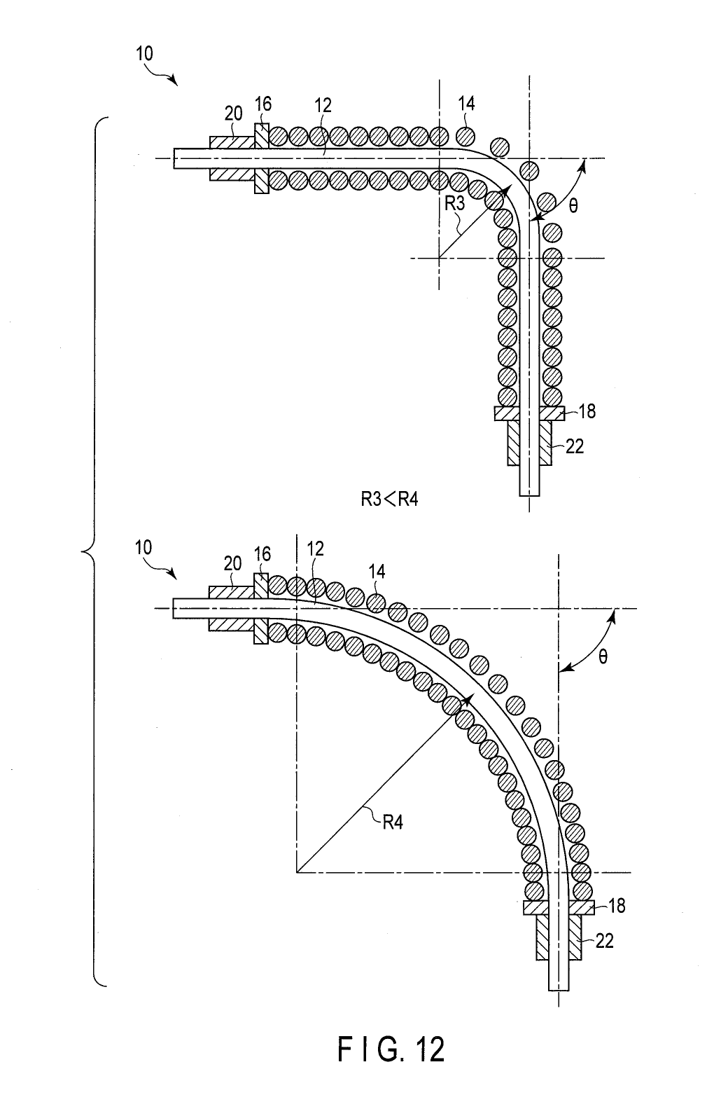

FIG. 12 shows a state in which the variable stiffness unit 10 shown in FIG. 11 is bent with different curvature radii. In FIG. 12, the variable stiffness unit 10 is drawn long in order to exaggerate the difference between the states. The bending angle θ of the core wire 12 is the same in both of the states shown in the upper part and the lower part of FIG. 12. However, the core wire 12 is bent with a curvature radius R3 in the state shown in the upper part of FIG. 12, while the core wire 12 is bent with a curvature radius R4 larger than the curvature radius R3 in the state shown in the lower part of FIG. 12.

As described above, since the gap length L2 and the center-to-center distance d1 define the bending angle θ1 of the core wire 12, but are not related to the curvature radius of the core wire 12, it may be possible both that a relatively narrow range of the core wire 12 is bent with the small curvature radius R3 as shown in the upper part of FIG. 12, and that a relatively wide range of the core wire 12 is bent with the large curvature radius R4 as shown in the lower part of FIG. 12.

In other words, since the condition in which the variable stiffness unit 10 is stiffened depends on the bending angle θ1 of the core wire 12, but does not depend on the curvature radius, the variable stiffness unit 10 may be partially bent strongly. If the variable stiffness unit 10 is partially bent strongly (bent with a small curvature radius), there is a risk of damaging the built-in member of the flexible member in which the variable stiffness apparatus including the variable stiffness unit 10 is installed. This applies to the case where a short variable stiffness unit 10 is strongly bent (bent with a small curvature radius) in its entirety as well as to the case where the variable stiffness unit 10 is partially bent.

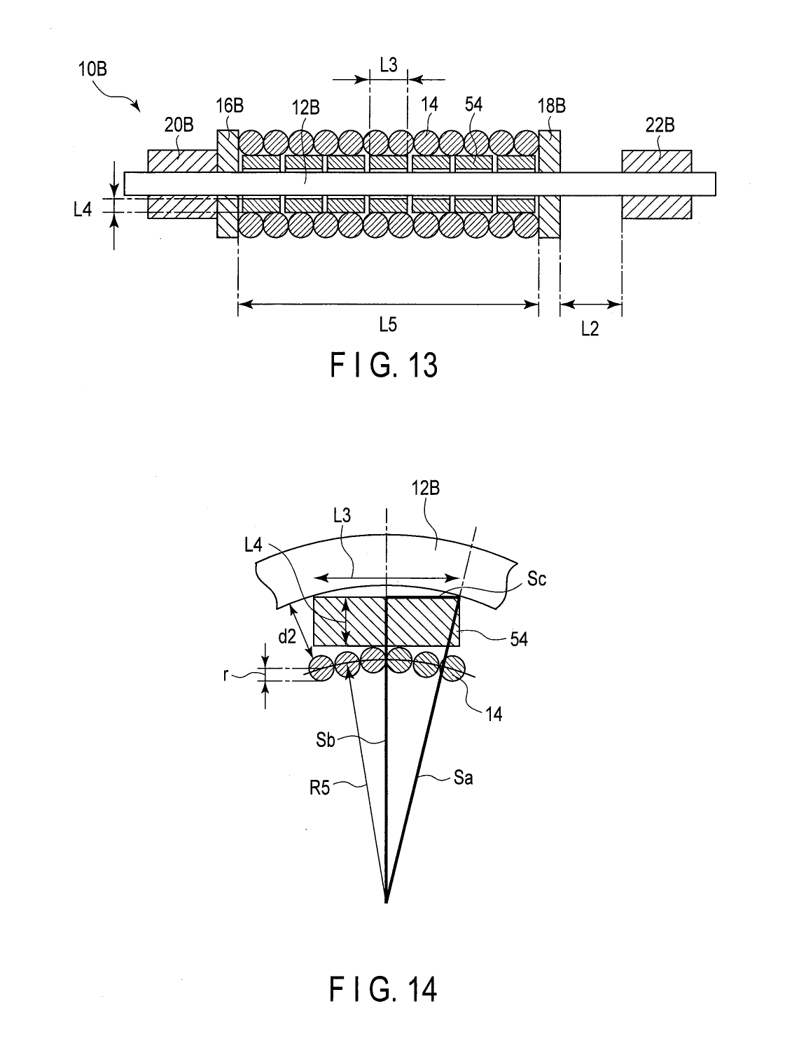

FIG. 13 shows a variable stiffness unit 10B according to a fifth embodiment. Similar to the variable stiffness unit 10 according to the first embodiment, the variable stiffness unit 10B includes a core wire 12B, a coil pipe 14, washers 16B and 18B, and fixing members 20B and 22B.

The core wire 12B of the variable stiffness unit 10B of the present embodiment has a smaller diameter than the core wire 12 of the variable stiffness unit 10 of the first embodiment. Therefore, a space is formed between the core wire 12B and the coil pipe 14. The washer 16B or 18B has a through hole with a smaller diameter than the through hole of the washer 16 or 18. The fixing member 20B or 22B has a smaller outer diameter than the fixing member 20 or 22.

The variable stiffness unit 10B further includes gap members 54 that maintain the distance between the coil pipe 14 and the core wire 12B when the coil pipe 14 is bent. Each gap member 54 has a pipe shape, and the core wire 12B extends through the gap members 54. In other words, the gap members 54 occupy the space formed between the core wire 12B and the coil pipe 14. Each gap member 54 may be made of, for example, a short metal pipe.

FIG. 14 shows an enlarged view of a part of the variable stiffness unit 10B shown in FIG. 13. In the state shown in FIG. 14, the variable stiffness unit 10B is bent with the bent core wire 12B being in contact with the edge portions of the gap member 54. As a result, the core wire 12B is not bent any further, and thus the inner curvature radius R5 of the variable stiffness unit 10B is restricted. That is, the variable stiffness unit 10B is not bent any further. In the present specification, the curvature radius R5 is referred to as the minimum curvature radius with which the bending stiffness of the variable stiffness unit 10B changes.

The bending stiffness of the variable stiffness unit 10B starts to increase when the variable stiffness unit 10B is bent at the bending angle θ1 of the core wire 12B, and becomes the maximum when the core wire 12B is bent at the minimum curvature radius.

The bending quantity of the variable stiffness unit 10B when the bending stiffness of the variable stiffness unit 10B starts to increase is changeable by adjusting the length L2 of the gap between the coil pipe 14 and the fixing member 20B or 22B when the variable stiffness unit 10B is in a linear state.

The inner curvature radius R5 of the variable stiffness unit 10B is given by the Equation (1) below. In the Equation (1) below, L3 is the length of the gap member 54, L4 is the thickness of the gap member 54, and d2 is the distance between the core wire 12B and the coil pipe 14. The length L3 of the gap member 54 is the dimension along the longitudinal direction of the core wire 12B, and the thickness L4 of the gap member 54 is the dimension along the radial direction of the core wire 12B. Herein, it is assumed that the core wire 12B is bent in an arc shape.

$$\begin{matrix}[ Equation ( 1 ) ] & \\ R 5 = \frac{L {3}^{2} + 4 L {4}^{2} - 4 d {2}^{2}}{8 ( d 2 - L 4 )} & ( 1 )\end{matrix}$$

Equation (1) is obtained as follows. In FIG. 14, the lengths of the sides Sa, Sb, and Sc of the triangle obtained by connecting the middle point of the gap member in the length direction, the virtual bending center point, and the contact point of the core wire 12B and the gap member 54 are considered. The length of the side Sa is R5+d2, the length of the side Sb is R5+L4, and the length of the side Sc is (L3)/2. Based on the Pythagorean theorem, (the length of the side Sb)²+(the length of the side Sc)²=(the length of the side Sa)². Equation (1) is obtained by substituting the aforementioned lengths of the sides Sa, Sb, and Sc into this equation and deforming the equation so as to solve the equation for R5.

It should be noted that Equation (1) is an equation assuming that the strand diameter of the coil pipe 14 is sufficiently smaller than the thickness L4 of the gap member 54. If the strand diameter of the coil pipe 14 is large, so that this assumption does not apply, the equation below is obtained. Regarding the strand diameter of the coil pipe 14 is r, the length of the side Sa is R5+d2+r/2, the length of the side Sb is R5+L4+r/2, and the length of the side Sc is (L3)/2. By substituting these lengths into the equation of the Pythagorean theorem and solving the equation similarly to the above, the following Equation (2) is obtained:

$$\begin{matrix}[ Equation ( 2 ) ] & \\ R 5 = \frac{L {3}^{2} + 4 L 4 ( L 4 + r ) - 4 d 2 ( d 2 + r )}{8 ( d 2 - L 4 )} & ( 2 )\end{matrix}$$

As understood from the Equations (1) and (2), the minimum curvature radius R5 with which the bending stiffness of the variable stiffness unit 10B changes is determined based on the dimensions of the gap member 54. Specifically, the minimum curvature radius R5 with which the bending stiffness of the variable stiffness unit 10B changes is determined based on the dimensions of the gap member 54, the outer diameter of the core wire 12B, and the inner diameter of the coil pipe 14. In other words, the gap member 54 serves to restrict the minimum curvature radius with which the bending stiffness changes.

In the variable stiffness unit 10B, it is possible to determine the minimum curvature radius with which the bending stiffness of the variable stiffness unit 10B changes. This enables preventing the built-in member of the flexible member from being damaged by excessive bending.

In the variable stiffness unit 10B, if the gap between the gap members 54 is too large, the core wire 12B may be strongly bent at a portion where the gap members 54 are absent. In order to avoid such a situation, the variable stiffness unit 10B is configured so that the gap members 54 come into contact with each other when the stiffness of the variable stiffness unit 10B changes.

With such a configuration, the gap members 54 come into contact with each other in a bent state in which the stiffness of the variable stiffness unit 10B changes, namely, in a state in which the core wire 12B is bent at the bending angle θ1. Accordingly, the gap members 54 are present in the entire length between the washers 16B and 18B with no gap between the gap members 54. As a result, the core wire 12B is prevented from being bent strongly at a specific portion.

In order to satisfy the above configuration, the dimensions of the coil pipe 14 and the gap member 54 are set so that the curvature radius R5′ when the portion between the gap members 54 having a gap L6 is bent until adjacent gap members 54 come into contact with each other is equal to or larger than the minimum curvature radius to be set.

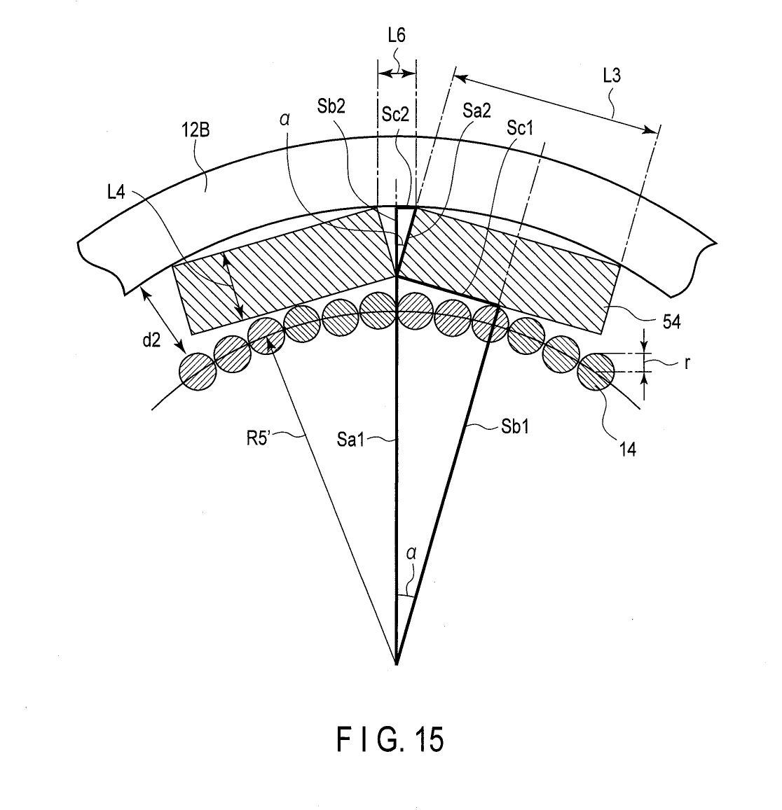

FIG. 15 shows an enlarged view of a part of the variable stiffness unit 10B shown in FIG. 13. In the state shown in FIG. 15, the variable stiffness unit 10B is bent in a state in which the bent core wire 12B is in contact with the edge portions of the gap members 54, and in which the adjacent gap members 54 are in contact with each other. A triangle having three sides Sa1, Sb1, and Sc1 and a triangle having three sides Sa2, Sb2, and Sc2 shown in FIG. 15 are assumed.

Focusing on the lower large triangle, the length of the side Sb1 is R5′+r/2, and the length of the side Sc1 is (L3)/2. Based on the Pythagorean theorem, the length of the side Sa1 is expressed by the following Equation (3):

$$\begin{matrix}[ Equation ( 3 ) ] & \\ The length of the side Sa 1 = \sqrt{{( R {5}^{′} + \frac{r}{2} )}^{2} + {( \frac{L 3}{2} )}^{2}} & ( 3 )\end{matrix}$$

Based on sin α=(the length of the side Sc1)/(the length of the side Sa1), the following Equation (4) is obtained:

$$\begin{matrix}[ Equation ( 4 ) ] & \\ sin α = \frac{L 3}{2} \times {( {( R {5}^{′} + \frac{r}{2} )}^{2} + {( \frac{L 3}{2} )}^{2} )}^{- 1 / 2} & ( 4 )\end{matrix}$$

Focusing on the upper small triangle, since L6=2×L4×sin α, the following Equation (5) is obtained:

$$\begin{matrix}[ Equation ( 5 ) ] & \\ L 6 = L 3 \times L 4 \times {( {( R {5}^{′} + \frac{r}{2} )}^{2} + {( \frac{L 3}{2} )}^{2} )}^{- 1 / 2} & ( 5 )\end{matrix}$$

Since the curvature radius R5′, which is determined by bending the variable stiffness unit 10B until the adjacent gap members 54 come into contact with each other, only has to be larger than the minimum curvature radius R5 to be set, in other words, to satisfy R5′>R5, the Equation (6) below only has to be satisfied. Here, n indicates the number of the gap members 54 included in the area of the length L5 (see FIG. 13) of the entire coil pipe 14 (namely, the total number of the gap members 54), and k indicates the number of gap members 54 included in the bent area in the area of the length L5 of the entire coil pipe 14. When the coil pipe 14 is entirely bent, k is equal to n.

$$\begin{matrix}[ Equation 6 ] & \\ ( n - 1 ) \times L 6 < k \times L 3 \times L 4 \times {( {( R 5 + \frac{r}{2} )}^{2} + {( \frac{L 3}{2} )}^{2} )}^{- 1 / 2} & ( 6 )\end{matrix}$$

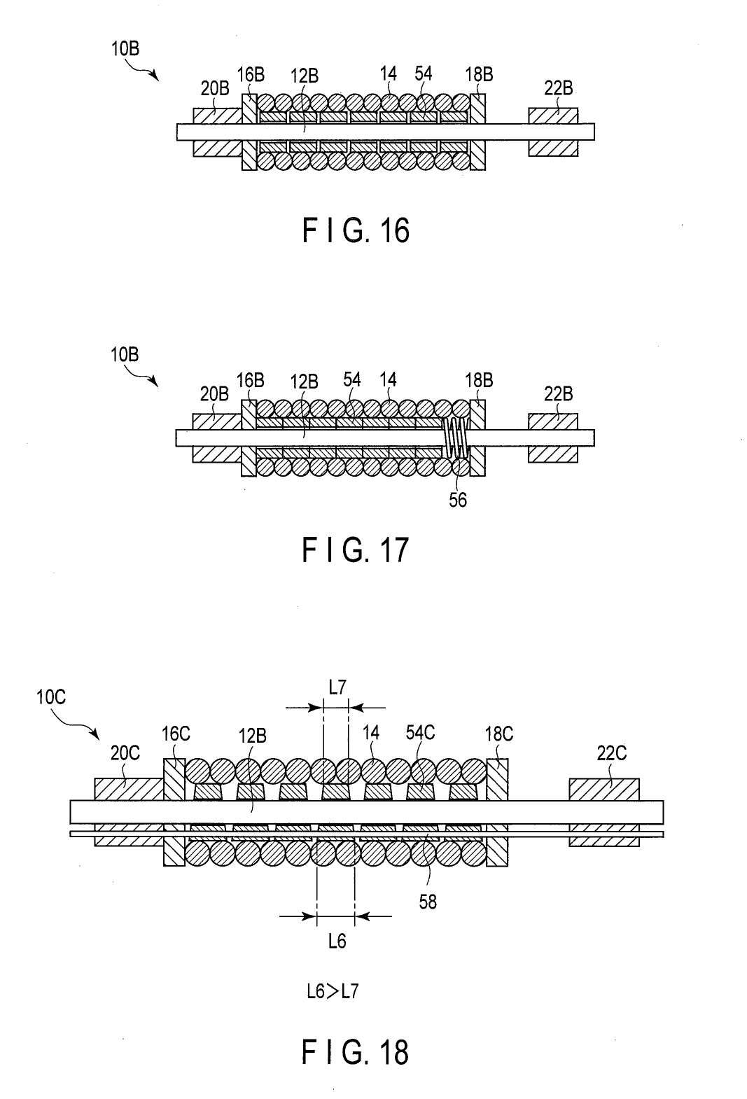

In a configuration example of the variable stiffness unit 10B that satisfies the above conditions, as shown in FIG. 16, the number of gap members 54 arranged inside the coil pipe 14 is sufficient so as not to provide a sufficient interval that allows the core wire 12B to be strongly bent at a specific portion.

In another configuration example of the variable stiffness unit 10B that satisfies the above conditions, as shown in FIG. 17, an urging member such as a coil spring 56 that urges the gap members 54 in the longitudinal direction of the core wire 12B is arranged inside the coil pipe 14. As a result, the gap members 54 are kept to be always in contact with each other.

### Sixth Embodiment

FIG. 18 shows a variable stiffness unit 10C according to a sixth embodiment. Similar to the variable stiffness unit 10B according to the fifth embodiment, the variable stiffness unit 10C includes a core wire 12B, a coil pipe 14, washers 16C and 18C, and fixing members 20C and 22C.

The core wire 12B extends through the washers 16C and 18C. The fixing members 20C and 22C are respectively fixed to the end portions of the core wire 12B. Similar to the fixing members 20B and 22, at least one of the fixing members 20C and 22C is releasable from being fixed to the core wire 12B, and may be movable along the core wire 12B if released from being fixed.

The variable stiffness unit 10C further includes gap members 54C that maintain the distance between the coil pipe 14 and the core wire 12B when the coil pipe 14 is bent. Each gap member 54C has a pipe shape, and the core wire 12B extends through the gap members 54C. Each gap member 54C has the maximum length L6 at a position on its periphery and the minimum length L7 at a position on its periphery located on the opposite side from the position of the length L6. Accordingly, the length of each gap member 54C is continuously different depending on the angular direction around the core wire 12B. Each gap member 54C has a symmetrical shape with respect to a plane perpendicular to its central axis. In other words, each gap member 54C is made of a pipe which is cut so that its cross section along the axis is trapezoidal.

The gap members 54C are aligned so as to have the same length in the same angular direction around the core wire 12B. In order to prevent rotation of the gap members 54C around the core wire 12B, a rotation preventing wire 58 extends through all the gap members 54C, the washers 16C and 18C, and the fixing members 20C and 22C. The rotation preventing wire 58 may be, for example, fixed to one of the fixing members 20C and 22C.

In the variable stiffness unit 10C, the length of each gap member 54C is different depending on the same angular direction around the core wire 12B. Thus, the minimum curvature radius with which the bending stiffness of the variable stiffness unit 10C changes is changed depending on the bending direction of the variable stiffness unit 10C.

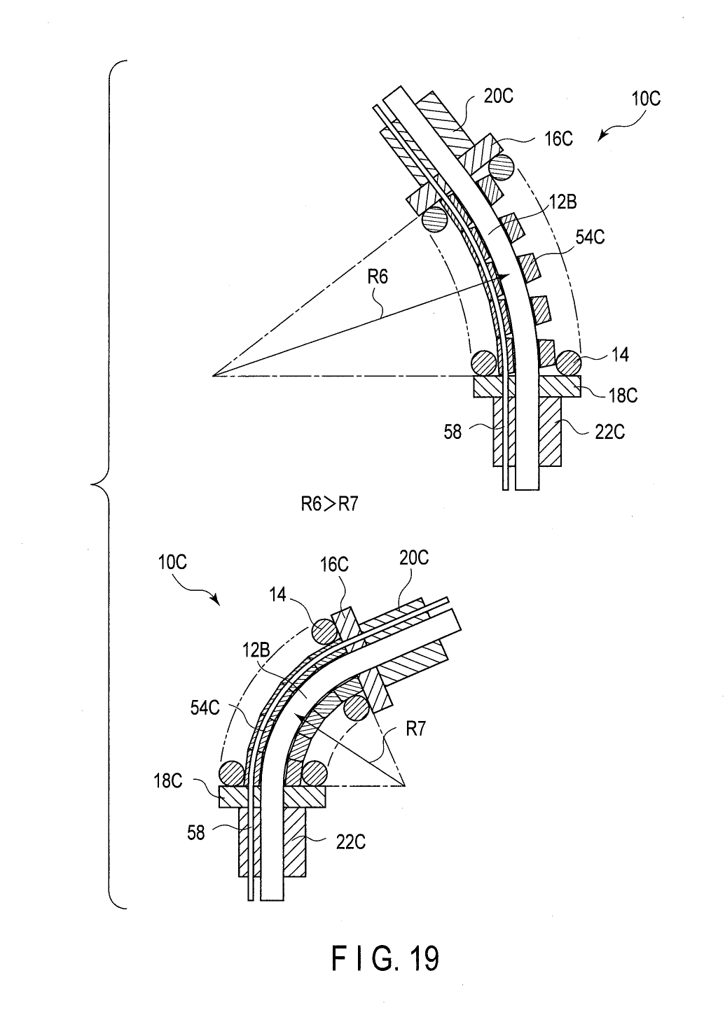

FIG. 19 shows a state in which the variable stiffness unit 10C is bent in different bending directions. The upper part of FIG. 19 shows a state in which the variable stiffness unit 10C is bent in a state in which the portions of the gap members 54C having the maximum length L6 face inward. In this state, the variable stiffness unit 10C is bent with a minimum curvature radius R6 with which the bending stiffness of the variable stiffness unit 10C changes.

In contrast, the lower part of FIG. 19 shows a state in which the variable stiffness unit 10C is bent in a state in which the portions of the gap members 54C having the minimum length L7 face inward. In this state, the variable stiffness unit 10C is bent with a minimum curvature radius R7 with which the bending stiffness of the variable stiffness unit 10C changes. The curvature radius R7 is smaller than the curvature radius R6.

In this manner, the bending quantity of the variable stiffness unit 10C when the variable stiffness unit 10C is bent with the minimum curvature radius with which the bending stiffness of the variable stiffness unit 10C changes is at minimum when the variable stiffness unit 10C is bent in the state in which the portions of the gap members 54C having the maximum length L6 face inward, and is at maximum when the variable stiffness unit 10C is bent in the state in which the portions of the gap members 54C having the minimum length L7 face inward.

When the variable stiffness unit 10C is bent in a state in which the portions of the gap members 54C between the portions having the maximum length L6 and the portions having the minimum length L7 face inward, the bending quantity of the variable stiffness unit 10C when the variable stiffness unit 10C is bent with the minimum curvature radius with which the bending stiffness of the variable stiffness unit 10C changes is intermediate.

Accordingly, in the variable stiffness unit 10C, the minimum curvature radius with which the bending stiffness of the variable stiffness unit 10C changes is different depending on the bending direction. In other words, the variable stiffness unit 10C has anisotropy for the minimum curvature radius with which the bending stiffness of the variable stiffness unit 10C changes.

The fact that the variable stiffness unit 10C has anisotropy for the minimum curvature radius with which the bending stiffness changes is useful for the insertion operation of the insertion section of the endoscope in which the variable stiffness unit 10C is installed.

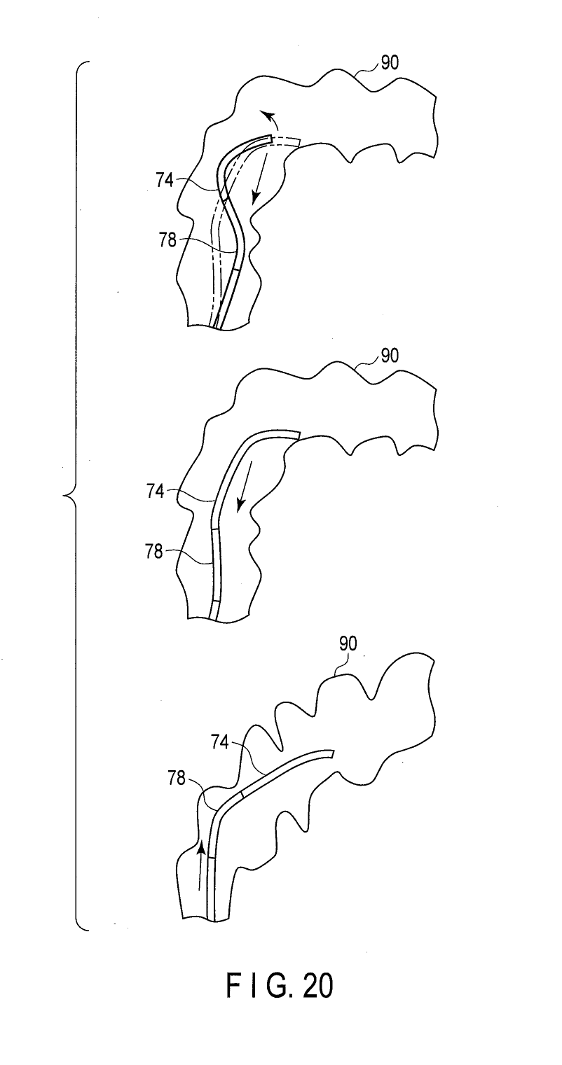

FIG. 20 shows a state in which the flexible tube 74 of the endoscope is inserted in the large intestine 90. In the insertion operation of the flexible tube 74 of the endoscope, when passing the flexible tube 74 through a largely bent portion of the large intestine 90, the flexible tube 74 is brought forward to the back of the large intestine 90 by hooking the distal end of the flexible tube 74 on the intestine tract of the large intestine 90 to pull it closer.

The insertion operation of the flexible tube 74 of the endoscope will be described below, assuming that the large intestine 90 is greatly bent rightward as shown in FIG. 20.

The upper part of FIG. 20 shows the insertion operation of the flexible tube 74 in the endoscope in which a variable stiffness unit without anisotropy for the minimum curvature radius with which the bending stiffness changes is installed in the passive bendable section 78 of the flexible tube 74. Herein, it is assumed that the minimum curvature radius with which the bending stiffness of the variable stiffness unit changes is an intermediate value between the maximum value R6 and the minimum value R7 of the minimum curvature radius with which the bending stiffness of the variable stiffness unit 10C changes.

In the operation of hooking the distal end of the flexible tube 74 on the intestine tract of the large intestine 90 to pull it closer, it is desirable that the passive bendable section 78 of the flexible tube 74 is not greatly bent leftward in FIG. 20.

The upper part of FIG. 20 shows a state in which the distal end of the flexible tube 74 receives a force from the intestine tract of the large intestine 90, and thus the passive bendable section 78 of the flexible tube 74 is unfortunately greatly bent leftward in FIG. 20, so that the distal end of the flexible tube 74 is unhooked from the intestine tract of the large intestine 90, which leads to failure in pulling the intestine tract of the large intestine 90 closer.

The middle part and the lower part of FIG. 20 show the insertion operation of the flexible tube 74 in the endoscope in which a variable stiffness unit 10C according to the present embodiment is installed in the passive bendable section 78 of the flexible tube 74. Herein, for example, it is assumed that the orientation of the flexible tube 74 is adjusted so that the minimum curvature radius with which the bending stiffness of the variable stiffness unit 10C changes with respect to the leftward bending in FIG. 20 is the maximum value, and the minimum curvature radius with which the bending stiffness of the variable stiffness unit 10C changes with respect to the rightward bending in FIG. 20 is the minimum value.

The middle part of FIG. 20 shows a state in which the passive bendable section 78 of the flexible tube 74 is not bent leftward in FIG. 20 any further, although the distal end of the flexible tube 74 receives a force from the intestine tract of the large intestine 90. In this state, the intestine tract of the large intestine 90 may be pulled closer in a preferable manner.

In the operation of bringing the flexible tube 74 forward after pulling the intestine tract of the large intestine 90 closer, it is desirable that the passive bendable section 78 of the flexible tube 74 is greatly bent rightward in FIG. 20. The orientation of the flexible tube 74 is adjusted so that the passive bendable section 78 of the flexible tube 74 may be greatly bent rightward in FIG. 20. The lower part of FIG. 20 shows a state in which the flexible tube 74 is brought forward to the back of the large intestine 90 after pulling the intestine tract of the large intestine 90 closer.

As described above, the variable stiffness unit 10C having anisotropy for the minimum curvature radius with which the bending stiffness changes is useful for the insertion operation of the insertion section of the endoscope in which the variable stiffness unit 10C is installed.

### Seventh Embodiment

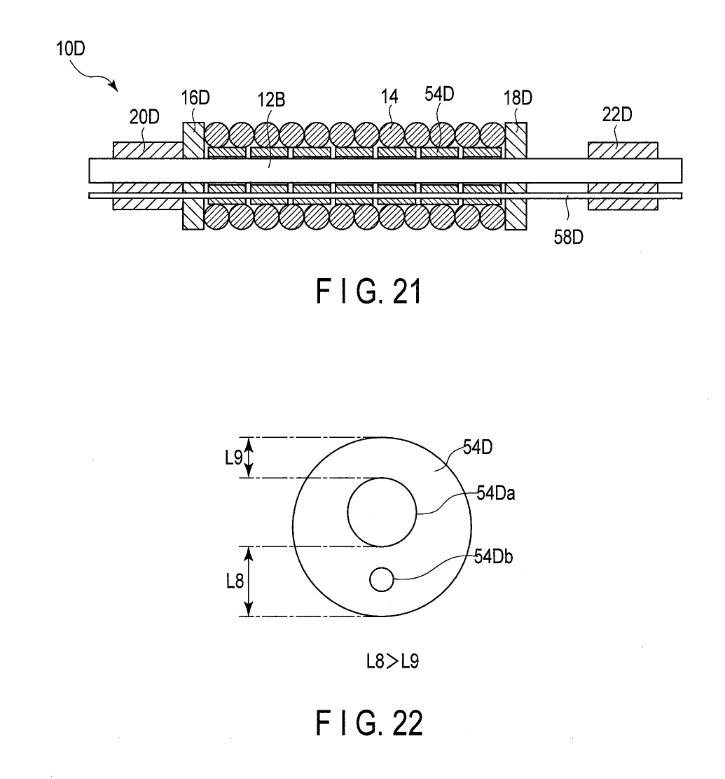

FIG. 21 shows a variable stiffness unit 10D according to a seventh embodiment. Similar to the variable stiffness unit 10B according to the fifth embodiment, the variable stiffness unit 10D includes a core wire 12B, a coil pipe 14, washers 16D and 18D, and fixing members 20D and 22D.

The core wire 12B extends through the washers 16D and 18D. The fixing members 20D and 22D are respectively fixed to the end portions of the core wire 12B. Similar to the fixing members 20 and 22, at least one of the fixing members 20D and 22D is releasable from being fixed to the core wire 12B, and may be movable along the core wire 12B if released from being fixed.

The variable stiffness unit 10D further includes gap members 54D that maintain the distance between the coil pipe 14 and the core wire 12B when the coil pipe 14 is bent. Each gap member 54D has an eccentric pipe shape, and the core wire 12B extends through the gap members 54D.

In order to prevent rotation of the gap members 54D around the core wire 12B, a rotation preventing wire 58D extends through all the gap members 54D, the washers 16D and 18D, and the fixing members 20D and 22D. The rotation preventing wire 58D may be, for example, fixed to one of the fixing members 20D and 22D.

As shown in FIG. 22, each gap member 54D has a through hole 54Da through which the core wire 12B passes, and a through hole 54Db through which the rotation preventing wire 58D passes. The through hole 54Da is deviated from the center of the gap member 54D. Therefore, each gap member 54D has the maximum thickness L8 and the minimum thickness L9 on a straight line passing through the center of the gap member 54D and the center of the through hole 54Da. Therefore, the thickness of each gap member 54D is continuously different depending on the angular direction around the core wire 12B.

The gap members 54D are aligned so as to have the same thickness in the same angular direction around the core wire 12B. The rotation preventing wire 58D extends through all the gap members 54D, the washers 16D and 18D, and the fixing members 20D and 22D, and thus the rotation of the gap members 54D around the core wire 12B is prevented. The rotation preventing wire 58D may be, for example, fixed to one of the fixing members 20D and 22D.

In the variable stiffness unit 10D, the thickness of each gap member 54D is different depending on the angular direction around the core wire 12B. Thus, the minimum curvature radius with which the bending stiffness of the variable stiffness unit 10D changes is changed depending on the bending direction of the variable stiffness unit 10D.

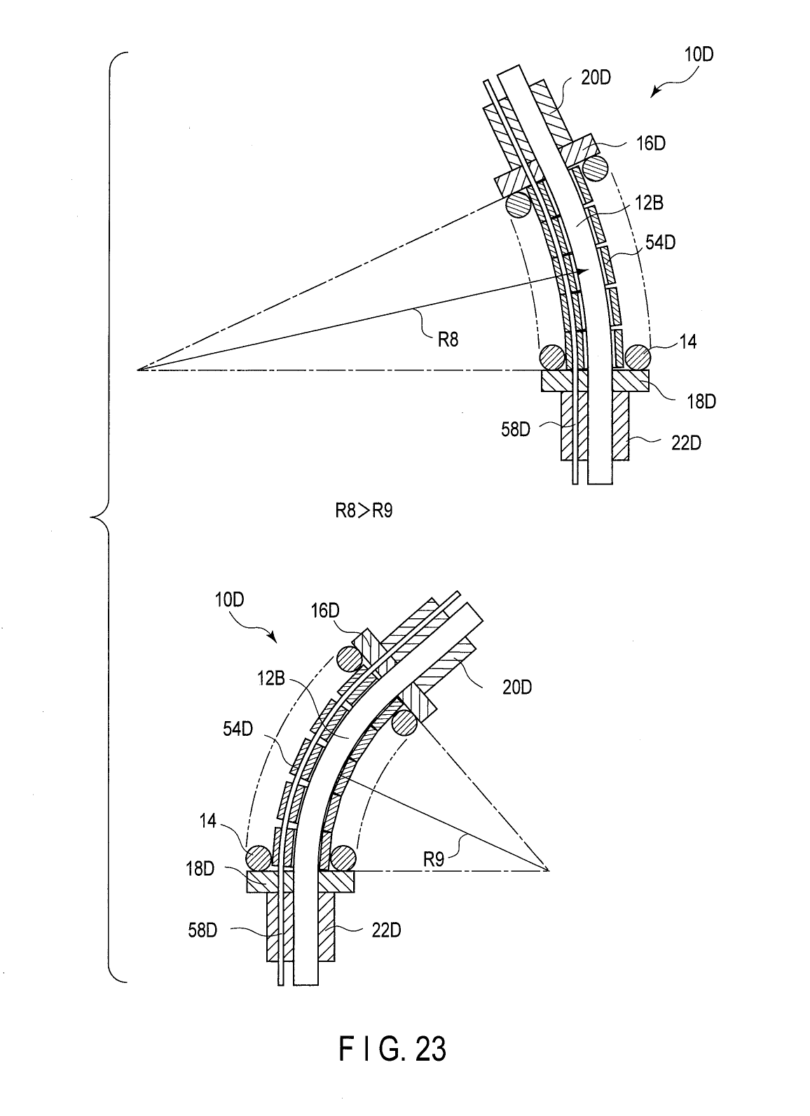

FIG. 23 shows a state in which the variable stiffness unit 10D is bent in different bending directions. The upper part of FIG. 23 shows a state in which the variable stiffness unit 10D is bent so that the portions of the gap members 54D having the maximum thickness L8 face inward. In this state, the variable stiffness unit 10D is bent with the minimum curvature radius R8 with which the bending stiffness of the variable stiffness unit 10D changes.

In contrast, the lower part of FIG. 23 shows a state in which the variable stiffness unit 10D is bent so that the portions of the gap members 54D having the minimum thickness L9 face inward. In this state, the variable stiffness unit 10D is bent with the minimum curvature radius R9 with which the bending stiffness of the variable stiffness unit 10D changes. The curvature radius R9 is smaller than the curvature radius R8.

In this manner, the bending quantity of the variable stiffness unit 10D when the variable stiffness unit 10D is bent with the minimum curvature radius with which the bending stiffness of the variable stiffness unit 10D changes is at minimum when the variable stiffness unit 10D is bent in the state in which the portions of the gap members 54D having the maximum thickness L8 face inward, and is at maximum when the variable stiffness unit 10D is bent in the state in which the portions of the gap members 54D having the minimum thickness L9 face inward.

When the variable stiffness unit 10D is bent in a state in which the portions of the gap members 54D between the portions having the maximum thickness L8 and the portions having the minimum thickness L9 face inward, the bending quantity of the variable stiffness unit 10D when the variable stiffness unit 10D is bent with the minimum curvature radius with which the bending stiffness of the variable stiffness unit 10D changes is intermediate.

Accordingly, in the variable stiffness unit 10D, the minimum curvature radius with which the bending stiffness of the variable stiffness unit 10D changes is different depending on the bending direction. In other words, the variable stiffness unit 10D has anisotropy for the minimum curvature radius with which the bending stiffness of the variable stiffness unit 10D changes.

The fact that the variable stiffness unit 10D has anisotropy for the minimum curvature radius with which the bending stiffness changes is useful for the insertion operation of the insertion section of the endoscope in which the variable stiffness unit 10D is installed, similarly to the variable stiffness unit 10C of the sixth embodiment.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A variable stiffness apparatus that is installed inside a flexible member to provide different levels of stiffness to the flexible member, the variable stiffness apparatus comprising:
at least one variable stiffness unit, each of the at least one variable rigidity unit including:
a flexible coil pipe;
a core wire extending inside the coil pipe;
a pair of fixing members arranged on both sides of the coil pipe and fixed to the core wire; and
an adjustment mechanism that adjusts at least one gap between the coil pipe and at least one of the fixing members.

2. The variable stiffness apparatus according to claim 1, wherein bending stiffness of the variable stiffness apparatus increases when the core wire is stretched as the coil pipe is bent and a tensile stress to the core wire increases as the core wire is stretched.

3. The variable stiffness apparatus according to claim 1, wherein at least one of the pair of fixing members is releasable from being fixed to the core wire, and is movable along the core wire if released from being fixed.

4. The variable stiffness apparatus according to claim 1, wherein the adjustment mechanism comprises a pulling mechanism that pulls at least one of the pair of fixing members in a direction to move the pair of fixing members away from each other.

5. The variable stiffness apparatus according to claim 1, wherein each of the at least one variable stiffness unit further comprises gap members that maintain a distance between the coil pipe and the core wire when the coil pipe is bent.

6. The variable stiffness apparatus according to claim 5, wherein each of the gap members serves to restrict a minimum curvature radius with which bending stiffness changes.

7. The variable stiffness apparatus according to claim 6, wherein a length of each of the gap members is different depending on an angular direction around the core wire, and the minimum curvature radius with which the bending stiffness of the variable stiffness unit changes is different depending on a bending direction.

8. The variable stiffness apparatus according to claim 6, wherein a thickness of each of the gap members is different depending on an angular direction around the core wire, and the minimum curvature radius with which the bending stiffness of the variable stiffness unit changes is different depending on a bending direction.

9. The variable stiffness apparatus according to claim 1, comprising variable stiffness units arranged inside the flexible member along a longitudinal direction.

10. The variable stiffness apparatus according to claim 1, wherein the adjustment mechanism is capable of continuously changing a gap between the coil pipe and the fixing members, and stiffness of the variable stiffness apparatus changes when the core wire is bent at a specific bending angle or further.

11. An endoscope comprising the variable stiffness apparatus according to claim 1.

12. An endoscope comprising a flexible tube, the flexible tube including:
an active bendable section that is bendable by operation; and
a passive bendable section located closer to the hand side than the active bendable section,
the variable stiffness apparatus according to claim 1 being provided in the passive bendable section.
